(19) United States
(12) Patent Application Publication
(10) Pub. No.: US 2006/0188463 A1
(43) Pub. Date: Aug. 24, 2006

(54) Stable water-in-oil-in-water multiple emulsion system produced by hydrodynamic dual stabilization and a method for preparation thereof

(76) Inventors: Jin Woong Kim, Kyungki-do (KR); Sung Il Lee, Seoul (KR); Byung Guen Chae, Seoul (KR); Han Kon Kim, Kyungki-do (KR); Hak Hee Kang, Kyungki-do (KR); Ih Seop Chang, Kyungki-do (KR)

Correspondence Address: SUGHRUE MION, PLLC, 2100 PENNSYLVANIA AVENUE, N.W., SUITE 800, WASHINGTON, DC, 20037, US

(21) Appl. No.: 09/878,714

(22) Filed: Jun. 11, 2001

(30) Foreign Application Priority Data
Dec. 29, 2000 (KR) 2000-85163

(51) Int. Cl. A61K 8/73

(52) U.S. Cl. 424/70.13

(57) ABSTRACT

The present invention relates to a water-in-oil-in-water multiple emulsion system and a method for preparation thereof, characterized by hydrodynamically stabilizing the multiple emulsion system by using hydrodynamic dual stabilization (HDS) technology. The HDS technology hydrophobizes water molecules in the internal aqueous phase by using a hydrogen bonding inhibitor and by improving the aggregating force between water molecules in the internal aqueous phase by using a water molecule aggregating agent.

### BACKGROUND OF THE INVENTION

1. Field of the Invention

The present invention relates to a water-in-oil-in-water multiple emulsion system, and a method for preparing the same, with a greatly improved stability of the internal aqueous phase of the multiple emulsion system. In particular, the present invention relates to a method for preparing a water-in-oil-in-water multiple emulsion system with highly improved stability achieved through the following steps: 1) “hydrophobizing” (processing a hydrophilic material so as to increase its hydrophobicity) the internal aqueous phase by using Hydrodynamic Dual Stabilization (hereinafter referred to as HDS) technology of the present invention in order to maximize dispersion stability such that the internal aqueous phase stably interacts with the oil phase of the multiple emulsion; and, 2) re-emulsifying the water-in-oil emulsion from step one in order to block the outflow of the internal aqueous phase domain to the external aqueous phase in the resultant water-in-oil-in-water multiple emulsion system.

2. Description of the Related Art

Multiple emulsions can be of the water-in-oil-in-water or of the oil-in-water-in-oil form, but water-in-oil-in-water emulsions have been more widely applied to various fields. In drug delivery systems for example, the water-soluble active material(s) to be delivered is placed into the internal aqueous phase for chemical or physical release in target sites. This multiple emulsion is a unique system in which the water domain is formed inside the oil phase of multiple emulsion system.

Until recently, a water-in-oil-in-water multiple emulsion was prepared by either a phase-transition method or two-step consecutive emulsification method. The phase-transition method using bifunctional solubility of the surfactant itself is limited in scope of application because it yields an unstable multiple emulsion. Therefore, a two-step consecutive emulsification method for improving stability has recently been developed. This two-step consecutive emulsification method comprises the following two steps: 1) preparing a water-in-oil emulsion by using hydrophobic surfactants; and, 2) re-emulsifying the water-in-oil emulsion obtained in the above with a water phase containing a suitable amount of hydrophilic surfactants.

The efficacy of the two-step consecutive emulsification method is affected by the mixing ratio of the surfactants, the properties of the oil being used, mechanical shearing force, volume ratio of the phases and the introduction of water-soluble additives. When re-emulsifying the water-in-oil emulsion suitable surfactants must be chosen to prepare a stable multiple emulsion. It is commonly known that the hydrophilic-lypophilic balance (hereinafter referred to as HLB) value of surfactants is preferably between approximately three to approximately seven in a water-in-oil emulsion, and is preferably between about eight to about sixteen in water-in-oil-in-water emulsions. Choice of a suitable surfactant is also dependent upon oil properties, thus the optimal HLB value range of selected surfactant may vary from system to system.

Further, when preparing a multiple emulsion by selecting suitable surfactants, factors including mechanical shearing force and volume ratio of phase affect droplet size, dispersibility, yield of multiple emulsion and phase morphology of the final multiple emulsion. For optimal yield and stability suitable conditions must be determined experimentally, depending upon the field of application. Water-soluble materials, that which is to be delivered by the multiple emulsion, added to the internal or external aqueous phase of a multiple emulsion system may reduce or enhance stability of the multiple emulsion system. The addition of the water soluble material forms a concentration gradient between the internal and external aqueous phases and must be countered by the addition of a suitable non-active water-soluble material to the external aqueous phase in order to attenuate the concentration difference between the internal and external aqueous phases.

For the reasons discussed above the stability of a multiple emulsion system can be improved only within a narrow range by changes of thermodynamic phase composition and can be optimized only by laborious empirical determination. A system that both reduces the number of factors affecting the stability of the multiple emulsion system and results in greatly improved stability would therefore be desirable.

The invention described herein represents a multiple emulsion system that both greatly improves the stability of the multiple emulsion and reduces the number of variables that need to be considered when producing the multiple emulsion, as well as a method for producing the same. By using a hydrogen bonding inhibitor to hydrophobize water molecules in the internal aqueous phase and by using a water molecule aggregating agent to improve the aggregating force between water molecules, a hydrodynamically stabilized water-in-oil-in-water multiple emulsion systems is obtained.

### SUMMARY OF THE INVENTION

The present invention provides a multiple emulsion system with much-improved stability compared to the conventional multiple emulsion systems and a method for preparing the multiple emulsion system with improved stability.

The present invention is characterized by solving the fundamental problem of the thermodynamic instability of a water-in-oil-in-water multiple emulsion system through hydrodynamically stabilizing water in the internal aqueous phase. This method greatly improves upon the conventional methods of improving stability by obviating the need, among other considerations, to carefully select suitable surfactants and oils, adjust mechanical shearing force, control the volume ratio of phases, and introduce suitable water-soluble additives. This method also results in a more stable multiple emulsion system.

The instant invention is further characterized by a hydrodynamically stabilized water-in-oil-in-water multiple emulsion that includes, for example, a water-in-oil emulsion composition that is formed from an active material combined with water, an oil, and a hydrogen bonding inhibitor, a water molecule aggregating agent, and a hydrophobic surfactant. Preferably, the hydrogen bonding inhibitor is in an amount between about 0.01% and 5% by weight relative to the total weight of the water-in-oil-in-water multiple emulsion. Also, the water molecule aggregating agent is preferably in an amount between about 0.01% and 5% by weight relative to the total weight of the water-in-oil-in-water multiple emulsion. Next, the hydrophobic surfactant is in an amount between approximately 0.01% and approximately 10% by weight relative to the total weight of the water-in-oil-in-water multiple emulsion. Lastly, the water-in-oil multiple emulsion is reemulsified using a low mechanical shearing force in an aqueous phase by dissolving a hydrophilic surfactant to obtain the a water-in-oil-in-water multiple emulsion. The hydrophilic surfactant is preferably in an amount between about 0.01% and 10% by weight relative to the total weight of the water-in-oil-in-water multiple emulsion.

The hydrodynamically stabilized water-in-oil-in-water multiple emulsion of the instant invention preferably includes the hydrogen bonding inhibitor that is at least one of such inhibitors selected from the group that includes substances having a mutual attraction in at least two directions on the atoms of the water molecule, all water-soluble molecules, urea, thio-urea, alcohols, and low molecular weight amines. The water molecule aggregating agent is preferably at least one that is selected from the group including substances that are able to collect water molecules, cyclodextrine, derivatives of cyclodextrine, and analogues of cyclodextrine.

It is further preferably that the hydrophobic surfactant is at least one that is selected from the group of all hydrophobic surfactants having a hydrophilic-lypophilic balance value ranging between approximately 1 and approximately 7, and combinations, derivatives, and analogues thereof. The hydrophilic surfactant is preferably at least one that is selected from the group that includes all non-ionic surfactants having a hydrophilic-lypophilic balance value ranging between approximately 8 and approximately 20, all ionic surfactants with a hydrophilic-lypophilic balance of approximately 20 and greater, and combinations, derivatives, and analogues thereof.

The present invention contemplates a wide variety of active materials that can be combined, mixed, incorporated, and/or dissolved into the internal aqueous phase of the emulsion. For purposes of illustration but not limitation, the active material can be selected from the group that includes, a water-soluble reference material, such as kojic acid, which can be used for purposes of establishing a suitable multiple emulsion configuration according to the principles of the present invention. Additionally, for example without limitation, the active material can include cosmetics, vitamins, peptide compounds, amino acids, proteins, enzymes, extracts, medicaments, drugs, pharmacologically active agent, therapeutic agents, minerals, antibiotics, macrolide antibiotics, tetracycline HCl, minocyclin HCl, ampicillin, zentamycin HCl, and combinations, ingredients, derivatives, homologues, and analogues thereof.

In preferred variations of the instant invention, the multiple emulsion also includes a dispersion-stabilizing agent operative to increase the viscosity of the external aqueous phase and to increase the stability of the multiple emulsion. Preferably, the dispersion-stabilizing agent is at least one such agent that is selected from the group that includes, for example but not for imitation, a macromolecular substance that is capable of being dissolved in the aqueous phase of the emulsion. Such substances include, for example without limitation, gelatin, starch, xanthan gun, sodium polyacrylate, hydroxyethyl cellulose, carboxymethylcellulose, polypyrrolidone, polyvinyl alkyl ether, polyvinyl alcohol, random and block copolymers that conjugate a hydrophilic group to a main chain, and combinations, derivatives, and analogues thereof.

The instant invention also further contemplates a variety of methods for obtaining a multiple emulsion and emulsion system. For example, a multiple emulsion system as described above can be obtained by: a) hydrophobizing the composition by mixing the hydrogen bonding inhibitor and the water molecule aggregating agent; b) obtaining the stable water-in-oil emulsion by using mechanical shearing force after evenly mixing the hydrophobic water phase and the hydrophobic surfactant; c) re-emulsifying the water-in-oil emulsion by adding the hydrophilic surfactant; and d) stabilizing the multiple emulsion by increasing the viscosity of the external aqueous phase by adding the dispersion stabilizing agent. Any or all of the preceding variations and modification of the elements in the aforementioned embodiments may be employed for purposes of the methods according to the present invention.

These variations, modifications, and alterations of the various preferred embodiments and methods may be used either alone or in combination with one another as will become more readily apparent to those with skill in the art with reference to the following detailed description of the preferred embodiments and the accompanying figures and drawings.

### BRIEF DESCRIPTION OF DRAWINGS

The file of this patent contains at least one drawing and photograph executed in color. Copies of this patent with the color drawing and photographs will be provided by the U.S. Patent and Trademark Office upon request and payment of the necessary fee.

FIG. 1 is a color schematic drawing of the interfaces in water-in-oil-in-water multiple emulsion system obtained by HDS (hydrodynamic dual stabilization).

FIG. 2 is an optical microscope color image of a water-in-oil-in-water multiple emulsion obtained using the conventional method.

FIG. 3 is an optical microscope color image of a water-in-oil-in-water multiple emulsion obtained using HDS (adding approximately 0.5% by weight urea and approximately 1% by weight hydroxypropyl-beta-cyclodextrine).

FIG. 4 is an optical microscope color image of a water-in-oil-in-water multiple emulsion obtained by HDS in which approximately 1% by weight kojic acid is contained and stabilized in the internal aqueous phase.

### DETAILED DESCRIPTION OF THE INVENTION

According to the present invention, water molecules in the internal aqueous phase are hydrophobized by using a hydrogen bonding inhibitor, and the aggregating force between water molecules is improved by using an aggregating agent. This results in a hydrodynamically stabilized multiple emulsion system.

The method for preparing the multiple emulsion system of the present invention includes the following steps:

i) hydrophobizing water by mixing a hydrogen bonding inhibitor and a water molecule aggregating agent;

ii) obtaining a stable water-in-oil emulsion by using mechanical shearing force after evenly mixing the hydrophobic water phase and a hydrophobic surfactant;

iii) re-emulsifying the water-in-oil emulsion by adding a hydrophilic surfactant; and,

iv) stabilizing the multiple emulsion system by increasing the viscosity of the external aqueous phase with a dispersion stabilizing agent.

Water incorporates highly polar molecules. Further, water in the internal aqueous phase of a multiple emulsion system shares the same high polarity with water in the external aqueous phase. Therefore, improvement of stability by the conventional method is limited by the strong attraction between the water in the internal aqueous phase and the water in the external aqueous phase, even though surfactants are added and phase conditions are controlled. The present inventors were interested in overcoming the problem of water polarity by changing the polar properties of the water in the internal or the external phase.

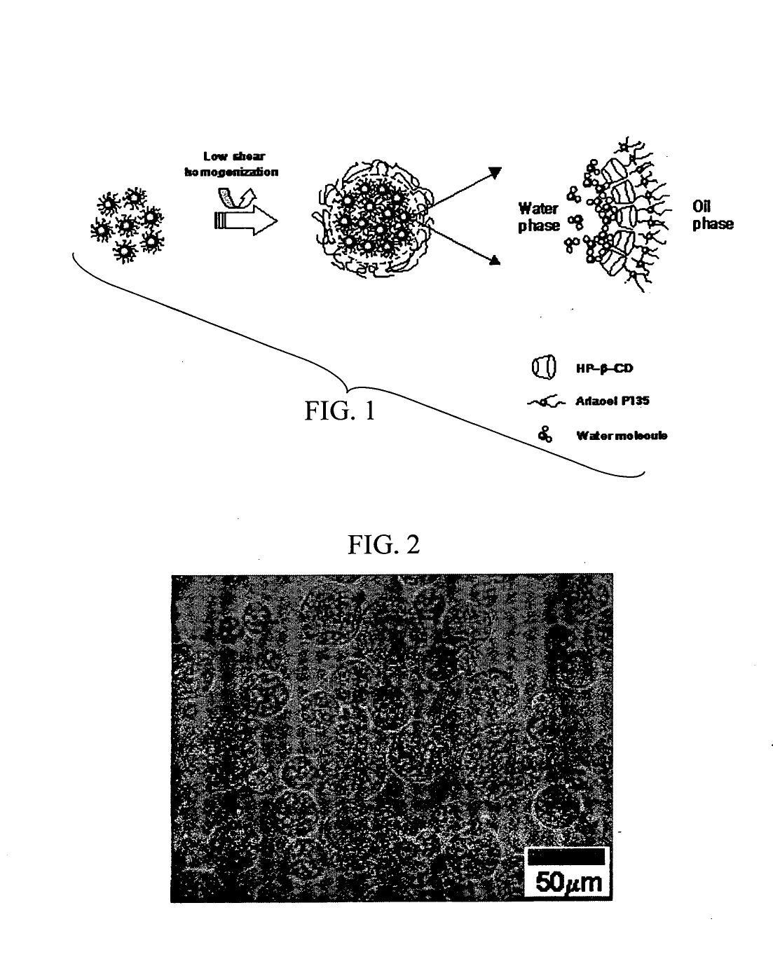

According to the present invention, compatibility of the internal aqueous phase with the oil phase can be improved, and the attraction between water in the internal phase and water in the external phase can be reduced. The HDS technology according to the present invention is based upon hydrodynamic hydrophobization of water in the internal aqueous phase and minimization of its fluidity. This is achieved by introducing a hydrogen bonding inhibitor and a water molecule aggregating agent into the water in the internal aqueous phase. The polarity of water is facilitated by the dissymmetrical linkage of the two hydrogen atoms to the one oxygen atom of a water molecule and by the extensive hydrogen bonding between water molecules. Introducing a hydrogen bonding inhibitor reduces the polarity of water. Water molecules with reduced polarity may then be changed to macromolecular form by secondary treatment with a water molecule aggregating agent. Therefore, the water phase is hydrophobized and its fluidity is minimized by treatment with a hydrogen bonding inhibitor and an aggregating agent, respectively. The resultant multiple emulsion system possesses excellent stability in the internal aqueous phase. These procedures are further illustrated in the diagrammatic representation depicted in FIG. 1.

When the water phase treated with the HDS is positioned onto the internal phase of a multiple emulsifying system, the stability of emulsion system is maximized in the following ways. First, unlike the conventional methods of simply decreasing the interfacial tension by using surfactants, the hydrophobization of the water phase improves the compatibility of two interfaces by creating a natural affinity between the water phase and the oil phase. The improved compatibility of the water of the internal phase and the oil phase results in a primary preparation of a water-in-oil emulsion with extremely small water domains, thereby contributing to an improved stability of the water-in-oil emulsion itself.

Second, the present invention solves the problem of interface weakening by outflow of the water into the external phase. The addition of a water aggregating substance decreases the diffusion of the internal aqueous phase into the oil is by minimizing the fluidity of water existing in the internal phase of a water-in-oil-in-water emulsion prepared through the secondary emulsifying procedure

Third, in drug delivery systems for example, the problems caused by the addition of water-soluble active materials (the active compound to be delivered) can be avoided using HDS technology without the use of other additives for improving the stability of the multiple emulsion. The water phase, hydromechanically stabilized by using a hydrogen bonding inhibitor and a water molecule aggregating agent as described above, is evenly mixed with a hydrophobic surfactant at a temperature higher than the melting points of either the water phase or the hydrophobic surfactant. A stable water-in-oil emulsion is then prepared by a strong mechanical shearing force, an external force for emulsifying through shearing and mixing particles in an aqueous or an oil phase using mechanical force. A typical example of an emulsifier is a homogenizer, which controls particle sizes and thus affects the degree of emulsification.

According to the present invention, the amount of hydrogen bonding inhibitor is preferably between approximately 0.01% and 5% by weight based on the total weight of the water-in-oil-in-water multiple emulsion system. It is preferable that the hydrogen bonding inhibitor has a mutual attraction in two or more directions on the hydrogen atom or oxygen atom of the water molecule. Examples of hydrogen bonding inhibitors include all water-soluble molecules such as urea, thio-urea, alcohols and low molecular weight amines.

It is preferable that the water molecule aggregating agent is able to collect water molecules. Examples of such water molecule aggregating agents include cyclodextrine and its derivatives and analogues. The amount of aggregating agent employed for the present invention is preferably between approximately 0.01% and 5% by weight based on the total weight of the water-in-oil-in-water multiple emulsion system.

Also, the amount of hydrophobic surfactant is preferably between approximately 0.01% and 10% by weight based on the total weight of the water-in-oil-in-water multiple emulsion system. The hydrophobic surfactant is selected from all hydrophobic surfactants having the HLB value within the range of approximately 1 and 7. Further, the water-in-oil emulsion obtained under the above delineated emulsification conditions is re-emulsified using low mechanical shearing force in an aqueous phase dissolving a hydrophilic surfactant to obtain a water-in-oil-in-water multiple emulsion. The hydrophilic surfactant is preferably between approximately 0.01% and 10% by weight based on the total weight of the water-in-oil-in-water multiple emulsion system. Such surfactants include all non-ionic surfactants within an HLB value range of between approximately 8 and 20, all ionic surfactants with an HLB value of approximately 20 or more, or combinations thereof.

Finally, according to the present invention, a dispersion-stabilizing agent may be added to the water-in-oil-in-water multiple emulsion in order to increase the viscosity of the external aqueous phase, resulting in a much-improved stability of the multiple emulsion. The dispersion-stabilizing agent employed in the present invention is preferably a macromolecule capable of being dissolved in aqueous phase. Examples of such dispersion stabilizing agents includes gelatin, starch, xanthan gun, sodium polyacrylate, hydroxyethyl cellulose, carboxymethylcellulose, polypyrrolidone, polyvinyl alkyl ether, polyvinyl alcohol, and random or block copolymers conjugating a hydrophilic group to a main chain and the like.

According to the present invention, the water-soluble active materials contained in the internal aqueous phase of water-in-oil-in-water multiple emulsion system include any active materials employed in cosmetics such as water-soluble ingredients of functional cosmetics including vitamin B, vitamin C and their derivatives; peptides compounds and proteins; enzymes; water-soluble plant extracts and active materials including the same; water-soluble functional active extracts; drugs or other pharmacologically active or otherwise therapeutic agents and the like. Water-soluble materials such as those described above introduced into a water-in-oil-in-water multiple emulsion system may result not only in stabilization of the emulsion but of the material itself and in the temporally controlled release of the material. Water-soluble antibiotics such as the macrolide-family of antibiotics, tetracycline HCl, minocyclin HCl, ampicillin, and zentamycin HCl are used for the primary treatment of infectious diseases. These antibiotics have a short half-life, however, and can be released too quickly to be optimally effective. A slow release rate is therefore a desirable property that enhances the efficacy of these antibiotics.

### EXEMPLARY EMBODIMENTS OF THE INVENTION

For purposes of illustration but not for purposes of limitation, the present invention is further explained by exemplary embodiments, and comparative and experimental examples.

### Example 1

### Preparation of Multiple Emulsion According to the Present Invention

A multiple emulsion stabilized using HDS was prepared by carrying out the following procedures. After hydrophobizing water by adding approximately 1% by weight of urea and approximately 2% by weight by of hydroxypropyl-beta-cyclodextrine (available from, for example, NIPPON Food Chemical Engineering Co., Ltd.), about 30% by weight of the hydrophobized water was melted, and was then gently stirred at a temperature of between approximately 70 and 75 degrees Celsius (Mixture A). The degree of hydrophobicity of water was determined by measuring the interface tension of the water. Then, approximately 15% by weight of mineral oil (LP70™, Witco Co., U.S.) dissolving approximately 1.5% by weight of hydrophobic surfactant and PEG-30 dipolyhydroxystearate (available from, for example, Arlacel P135™, ICI Company, Great Britain) were melted, stirring gently at a temperature of between approximately 70 and 75 degrees Celsius (Mixture B). Then, mixture A and B were mixed violently for 5 minutes using a homogenizer of between approximately 7,000 and approximately 8,000 revolutions per minute (hereafter also referred to as “rpm”) and then cooled at between approximately 50 and 60 degrees Celsius. A highly stable water-in-oil emulsion was obtained.

Next, the water phase dissolved in approximately 0.5% by weight of poloxamer 407 (available from, for example, Synperonic PE/F 127™, ICI Co.), was added slowly to water-in-oil emulsion obtained in the above procedure with stirring using a homogenizer of approximately 4,000 rpm at between approximately 50 and 60 degrees Celsius. Herein, the concentration was fixed to 50% by weight. Then approximately 1% by weight of xanthan gum solution (available from, for example, Kelfrol-F™, Kelco Co., U.S.) was added at an amount of approximately 10% by weight. The resulting water-in-oil-in-water multiple emulsion was stabilized and then cooled slowly at room temperature.

### Example 2

### Preparation of Multiple Emulsion Containing Kojic Acid in Internal Aqueous Phase

Kojic acid is used here as a water-soluble reference material and as a model of a water-soluble active material. A water-in-oil-in-water multiple emulsion was obtained using the same method as described in example 1 except that the water-in-oil emulsion was prepared after approximately 1% by weight kojic acid had been introduced into internal aqueous phase. At this time, heat treatment had to be carefully monitored in order that the active material was not modified by heat.

### Comparative Example 1

### Preparation of Multiple Emulsion with the Conventional Method

A conventional water-in-oil-in-water multiple emulsion was obtained using the same method as described in example 1 except that urea and hydroxypropyl-beta-cyclodextrine were not added. The multiple emulsions prepared by the conventional method and the present invention were used in the below experiments on stability.

### Experimental Example 1

### Stability of Multiple Emulsion

The multiple emulsions obtained in Examples 1, 2 and Comparative Example 1 were used in the following experiments and were prepared after MgSO₄ was introduced into each internal aqueous phase of each multiple emulsions. The resulting multiple emulsions were stored at approximately 4 degrees Celsius, approximately room temperature, approximately 30 degrees Celsius, approximately 37 degrees Celsius and approximately 45 degrees Celsius, respectively. The stability of the multiple emulsion obtained in Example 1 and the multiple emulsions obtained in Comparative Example 1 were evaluated by assaying changes in conductivity resulting from the outflow of electrolytes (MgSO₄) from the internal to the external aqueous phase. A sample of each multiple emulsion was taken once per day and its conductivity was measured in units of 1 μS/cm. The measured conductivity was compared with standard curves obtained by measuring conductivities of various known concentrations of MgSO₄, and the stabilities of multiple emulsions were determined by the following formula.

$$\begin{matrix}Stability of \\ Mutiple Emulsion ( \% )\end{matrix} = [ 1 - \frac{Measured Conductivity ( μ S \text{/} cm )}{Calculated Conductivity ( μ S \text{/} cm )} ]$$

It was found that about 15% of the electrolytes flowed out from the internal aqueous phase immediately after preparation of each of the multiple emulsions. It is thought that the initial outflow of electrolytes results from an unknown problem in the re-emulsification of the water-in-oil emulsion. After the loss of the initial 15% of electrolytes, the stability of the multiple emulsion obtained by HDS in Example 1 was maintained for several days at 70% or more, irrespective of storage conditions. The stability of multiple emulsion obtained by the conventional method of the prior art decreased to less than 50% under similar conditions. This result indicates that the procedure of hydrodynamically hydrophobizing the internal aqueous phase plays an important roles in improving the stability of a multiple emulsion.

The multiple emulsion obtained in Example 2, which contains kojic acid in internal aqueous phase, was stored at approximately 4 degrees Celsius, approximately room temperature, approximately 30 degrees Celsius, approximately 37 degrees Celsius and approximately 45 degrees Celsius, respectively. Changes in the color, odor and titer of the multiple emulsion were then assayed over time. The titer of the kojic acid in the multiple emulsion was determined by measuring the concentration of kojic acid using High Pressure Liquid Chromatography (hereafter referred to as HPLC). The color and odor of the emulsion were maintained for about two months and the titer after one month was 80% of the original titer.

### Experimental Example 2

### Analysis of Multiple Emulsion by Using Microscope

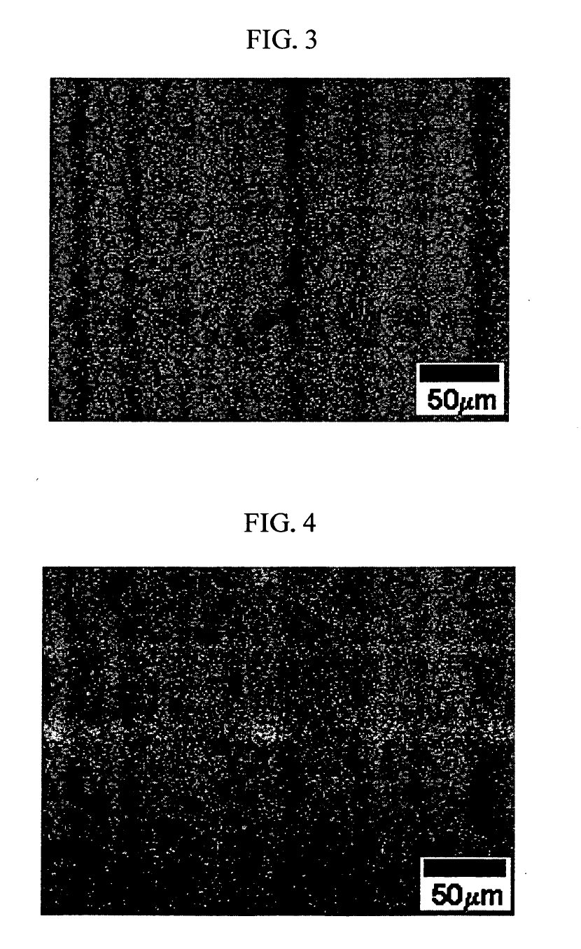

Droplet size and morphology of the multiple emulsions obtained in Example 1 and in Comparative Example 1 were photographed and measured using optical microscopy and the results are shown in FIG. 2 and in FIG. 3. Both the emulsions from Example 1 and from Comparative Example 1 include two interfaces of water-in-oil-in-water. The multiple emulsion obtained by HDS is different from other conventional multiple emulsions in that it has a monolithic morphology (FIG. 3), whereas the multiple emulsion obtained using conventional methods contains a large internal aqueous phase. Since the water in the internal aqueous phase is dispersed into small droplets, the ability of the internal aqueous phase to interact with the oil phase is greatly improved by hydrophobizing the water in the internal aqueous phase. The use of an internal aqueous phase containing urea and hyroxypropyl-beta-cyclodextrine reduced the interfacial tension by 20 mN/m or more when compared to using pure water as the internal aqueous phase. This result indicates that the reduction of polarity and the increased aggregation of water may be achieved through the introduction of urea and hydroxypropyl-beta-cyclodextrine.

A multiple emulsion both stabilizing and containing approximately 1% by weight of kojic acid was photographed using an optical microscope and the result is shown in FIG. 4. It is apparent that the morphology and droplet size of the HDS prepared multiple emulsion containing kojic acid was no different than the HDS prepared multiple emulsion without kojic acid, even though a relatively large amount of the water-soluble active material was added. Also, FIG. 4 shows that the water-soluble active material is stably positioned in the internal aqueous phase and that reduction of emulsion stability during its preparation does not occur. Droplet size of multiple emulsion is distributed evenly within the range of between approximately 2 and 20 μm. It is further shown that the degree of dispersion was somewhat reduced when the water in the internal aqueous phase was not treated with HDS. These results prove that, for emulsions that are not prepared using HDS technology, the water in the internal aqueous phase passes through the oil phase into the external aqueous phase, which indicates that internal phase of the conventional multiple emulsion becomes unstable.

As explained above, the present method for preparing multiple emulsion system using HDS is considerably different from other conventional methods. The method according to the present invention solves fundamental problems of instability inherent in conventional multiple emulsion systems. As such the present invention may extend the narrow use of conventional multiple emulsions to other fields and applications. In particular, multiple emulsions using HDS technology can be applied to cosmetics and to drug delivery systems to improve the stability and activity of those cosmetics, drugs or therapeutic agents whose activity is reduced in water. Also, multiple emulsions according to the present invention have excellent stability allowing for storage and can also be used to control the release rate of the water soluble active compound that may improve the efficacy of these compounds.

Although the exemplary embodiments of the present invention have been described in detail above, numerous alterations, modifications, and variations of the preferred embodiments disclosed herein will be apparent to those skilled in the art and they are all contemplated to be within the spirit and scope of the instant invention. For example, although specific embodiments, modifications, variations, and examples have been described in detail, those with skill in the art will understand that such can be modified to incorporate various types of substitute and/or additional substances, materials, elements, and relative arrangement of process steps, and quantities of described materials and substances for compatibility with the wide variety of possible active materials available and in use in the related industries. Accordingly, even though only few variations, modifications, and examples of the present invention are described herein, it is to be understood that the practice of such additional modifications and variations and the equivalents thereof, are within the spirit and scope of the invention as defined in the following claims.

What is claimed is:

1. A hydrodynamically stabilized water-in-oil-in-water multiple emulsion, comprising:
a water-in-oil emulsion composition formed from an active material combined with water, an oil, and hydrophobized with a hydrogen bonding inhibitor, a water molecule aggregating agent, and a hydrophobic surfactant.

2. The hydrodynamically stabilized water-in-oil-in-water multiple emulsion according to claim 1, wherein the hydrogen bonding inhibitor is in an amount between approximately 0.01% and approximately 5% by weight relative to the total weight of the water-in-oil-in-water multiple emulsion;
the water molecule aggregating agent is in an amount between approximately 0.01% and approximately 5% by weight relative to the total weight of the water-in-oil-in-water multiple emulsion; and
the hydrophobic surfactant is in an amount between approximately 0.01% and approximately 10% by weight relative to the total weight of the water-in-oil-in-water multiple emulsion; and
whereby the water-in-oil multiple emulsion is reemulsified using a low mechanical shearing force in an aqueous phase by dissolving a hydrophilic surfactant to obtain the a water-in-oil-in-water multiple emulsion; and
wherein the hydrophilic surfactant is in an amount between approximately 0.01% and approximately 10% by weight relative to the total weight of the water-in-oil-in-water multiple emulsion.

3. The hydrodynamically stabilized water-in-oil-in-water multiple emulsion according to claim 1, wherein the hydrogen bonding inhibitor is at least one selected from the group including substances that have a mutual attraction in at least two directions on the atoms of the water molecule, all water-soluble molecules, urea, thio-urea, alcohols, and low molecular weight amines.

4. The hydrodynamically stabilized water-in-oil-in-water multiple emulsion according to claim 1, wherein the water molecule aggregating agent is at least one selected from the group including substances that are able to collect water molecules, cyclodextrine, derivatives of cyclodextrine, and analogues of cyclodextrine.

5. The hydrodynamically stabilized water-in-oil-in-water multiple emulsion according to claim 1, wherein the hydrophobic surfactant is at least one selected from the group of all hydrophobic surfactants having a hydrophilic-lypophilic balance value ranging between approximately 1 and approximately 7, and combinations, derivatives, and analogues thereof.

6. The hydrodynamically stabilized water-in-oil-in-water multiple emulsion according to claim 1, wherein the hydrophilic surfactant is at least one selected from the group including all non-ionic surfactants having a hydrophilic-lypophilic balance value ranging between approximately 8 and approximately 20, all ionic surfactants with a hydrophilic-lypophilic balance of approximately 20 and greater, and combinations, derivatives, and analogues thereof.

7. The hydrodynamically stabilized water-in-oil-in-water multiple emulsion according to claim 1, wherein the active material is contained in the internal aqueous phase of the water-in-oil-in-water multiple emulsion and is at least one selected from the group including a water-soluble reference material, kojic acid, cosmetics, vitamins, peptide compounds, amino acids, proteins, enzymes, extracts, medicaments, drugs, pharmacologically active agent, therapeutic agents, minerals, antibiotics, macrolide antibiotics, tetracycline HCl, minocyclin HCl, ampicillin, zentamycin HCl, and combinations, ingredients, derivatives, and analogues thereof

8. The hydrodynamically stabilized water-in-oil-in-water multiple emulsion according to claim 1, further comprising:
a dispersion-stabilizing agent operative to increase the viscosity of the external aqueous phase and to increase the stability of the multiple emulsion.

9. The hydrodynamically stabilized water-in-oil-in-water multiple emulsion according to claim 8, wherein the dispersion-stabilizing agent is at least one selected from the group including a macromolecule capable of being dissolved in the aqueous phase, gelatin, starch, xanthan gun, sodium polyacrylate, hydroxyethyl cellulose, carboxymethylcellulose, polypyrrolidone, polyvinyl alkyl ether, polyvinyl alcohol, random and block copolymers that conjugate a hydrophilic group to a main chain, and combinations, derivatives, and analogues thereof.

10. A hydrodynamically stabilized water-in-oil-in-water multiple emulsion, consisting essentially of:
a water-in-oil emulsion composition formed from an active material combined with water, an oil, and a hydrogen bonding inhibitor, a water molecule aggregating agent, and a hydrophobic surfactant wherein the hydrogen bonding inhibitor is in an amount between approximately 0.01% and approximately 5% by weight relative to the total weight of the water-in-oil-in-water multiple emulsion;
the water molecule aggregating agent is in an amount between approximately 0.01% and approximately 5% by weight relative to the total weight of the water-in-oil-in-water multiple emulsion; and
the hydrophobic surfactant is in an amount between approximately 0.01% and approximately 10% by weight relative to the total weight of the water-in-oil-in-water multiple emulsion; and
whereby the water-in-oil multiple emulsion is reemulsified using a low mechanical shearing force in an aqueous phase by dissolving a hydrophilic surfactant to obtain the a water-in-oil-in-water multiple emulsion; and
wherein the hydrophilic surfactant is in an amount between approximately 0.01% and approximately 10% by weight relative to the total weight of the water-in-oil-in-water multiple emulsion.

11. A method for preparing the multiple emulsion of claim 8, comprising the steps of:
a) hydrophobizing the composition by mixing the hydrogen bonding inhibitor and the water molecule aggregating agent;
b) obtaining the stable water-in-oil emulsion by using mechanical shearing force after evenly mixing the hydrophobic water phase and the hydrophobic surfactant;
c) re-emulsifying the water-in-oil emulsion by adding the hydrophilic surfactant; and
d) stabilizing the multiple emulsion by increasing the viscosity of the external aqueous phase by adding the dispersion stabilizing agent.

12. A method for preparing a multiple emulsion, comprising the steps of:
a) selecting a composition of an active material combined with water and an oil;
b) hydrophobizing the composition by mixing a hydrogen bonding inhibitor and a water molecule aggregating agent;
c) obtaining a stable water-in-oil emulsion by using mechanical shearing force after evenly mixing the hydrophobic water phase and a hydrophobic surfactant;
d) re-emulsifying the water-in-oil emulsion by adding a hydrophilic surfactant; and
e) stabilizing the multiple emulsion by increasing the viscosity of the external aqueous phase with a dispersion stabilizing agent.

13. The method for preparing a multiple emulsion according to claim 12, further comprising the step of:
f) selecting the hydrogen bonding inhibitor for hydrophobizing the composition to have a mutual attraction in at least two directions on the atoms of the water molecule, and wherein the inhibitor is at least one selected from the group of water-soluble materials including urea, thio-urea, alcohols with low molecular weight, and amines with low molecular weight.

14. The method for preparing a multiple emulsion according to claim 12, wherein the water molecule aggregating agent is at least one selected from the group of compounds with ability to aggregate water molecules, cyclodextrine, derivatives of cyclodextrine, and analogues of cyclodextrine.

15. The method for preparing a multiple emulsion according to claim 12, wherein the hydrophobic surfactant is at least one selected from the group of surfactants including:
(a) hydrophobic surfactants having a hydrophilic-lypophilic balance value ranging between approximately 1 and approximately 7;
(b) non-ionic surfactants having a hydrophilic-lypophilic balance value ranging between approximately 8 and approximately 20; and
(c) ionic surfactants having a hydrophilic-lypophilic balance value ranging from approximately 20 and greater.

16. The method for preparing a multiple emulsion according to claim 12, wherein the dispersion stabilizing agent is at least one selected from the group including a macromolecule capable of being dissolved in the aqueous phase, gelatin, starch, xanthan gun, sodium polyacrylate, hydroxyethyl cellulose, carboxymethylcellulose, polypyrrolidone, polyvinyl alkyl ether, polyvinyl alcohol, random and block copolymers that conjugate a hydrophilic group to a main chain, and combinations, derivatives, and analogues thereof.

17. The method for preparing a multiple emulsion according to claim 12, wherein:
the hydrogen bonding inhibitor is in an amount between approximately 0.01% and approximately 5% by weight relative to the total weight of the water-in-oil-in-water multiple emulsion;
the water molecule aggregating agent is in an amount between approximately 0.01% and approximately 5% by weight relative to the total weight of the water-in-oil-in-water multiple emulsion;
the hydrophobic surfactant is in an amount between approximately 0.01% and approximately 10% by weight relative to the total weight of the water-in-oil-in-water multiple emulsion; and
the hydrophilic surfactant is in an amount between approximately 0.01% and approximately 10% by weight relative to the total weight of the water-in-oil-in-water multiple emulsion.

18. The method for preparing a multiple emulsion according to claim 17, wherein:
the dispersion stabilizing agent is in an amount between approximately 0.01% and 10% by weight relative to the total weight of the water-in-oil-in-water multiple emulsion.

19. The method for preparing a multiple emulsion according to claim 12, wherein the active material is contained in the internal aqueous phase of the water-in-oil-in-water multiple emulsion and is at least one selected from the group including a water-soluble reference material, kojic acid, cosmetics, vitamins, peptide compounds, proteins, amino acids, enzymes, extracts, medicaments, drugs, pharmacologically active agent, therapeutic agents, minerals, antibiotics, macrolide antibiotics, tetracycline HCl, minocyclin HCl, ampicillin, zentamycin HCl, and combinations, ingredients, derivatives, and analogues thereof

20. The method for preparing a multiple emulsion according to claim 12, wherein the dispersion-stabilizing agent is at least one selected from the group including a macromolecule capable of being dissolved in the aqueous phase, gelatin, starch, xanthan gun, sodium polyacrylate, hydroxyethyl cellulose, carboxymethylcellulose, polypyrrolidone, polyvinyl alkyl ether, polyvinyl alcohol, random and block copolymers that conjugate a hydrophilic group to a main chain, and combinations, derivatives, and analogues thereof.
